(19) United States
(12) Patent Application Publication
(10) Pub. No.: US 2014/0221429 A1
(43) Pub. Date: Aug. 7, 2014

(54) HETEROCYCLIC SCHIFF'S BASES AS NOVEL AND NEW ANTIGLYCATION AGENTS

(71) Applicants: Saud Ibrahim Al-Resayes, Riyadh (SA); Ismail Warad, Nablus (PS); Mohammed A. Al-Nuri, Nablus (PS); Muhammad Iqbal Choudhary, Karachi (PK); Atia-tul Wahab, Karachi (PK); Saima Rasheed, Karachi (PK)

(72) Inventors: Saud Ibrahim Al-Resayes, Riyadh (SA); Ismail Warad, Nablus (PS); Mohammed A. Al-Nuri, Nablus (PS); Muhammad Iqbal Choudhary, Karachi (PK); Atia-tul Wahab, Karachi (PK); Saima Rasheed, Karachi (PK)

(21) Appl. No.: 13/757,956

(22) Filed: Feb. 4, 2013

(51) Int. Cl. A61K 31/444

(52) U.S. Cl. CPC A61K 31/444
USPC 514/332

(57) ABSTRACT

The invention provides that heterocyclic Schiff bases posses protein antiglycation potential in hyperglycemia. The novel antiglycation agents 3-6 showed a moderate to potent antiglycation activity.

### BACKGROUND OF THE INVENTION

Glycation of proteins plays a role in the process of aging and other disorders, such as diabetes and related complications. It is a spontaneous non-enzymatic reaction, and its rate is accelerated in diabetic individuals due to hyperglycemia, though the reaction occurs in normal individuals as well but at a slower rate. Glycation of proteins in hyperglycemia leads to the formation of Advanced Glycation Endproducts (AGEs) through a complex reaction sequence. Preventing or slowing down the molecular processes of formation of AGEs is considered to be an important approach towards the treatment of late diabetic complications.

Glycation not only involves amino groups of proteins but it also leads to the chemical modifications of basic residues of lipids and nucleic acids. The initial step in protein glycation is the formation of Schiff base in which glucose reacts non-enzymatically with the amino groups of proteins, the labile Schiff base can rearrange itself to a more stable, irreversible configuration known as Amadori product, which ultimately give rise to a poorly characterized heterogeneous group of compounds called advance glycation end products.

The risk factors due to the accumulation and circulation of AGEs include diabetes-specific complications of the micro-vasculature (retinopathy, nephropathy, and neuropathy) and complications of the macro-vasculature systems (atherosclerosis leading to heart disease, stroke and peripheral vascular disease). A number of other problems which are associated with the glycation of biomolecules in hyperglycemic state include chronic vascular complications of diabetes, non-diabetic nephropathy, macrovascular disease, Alzheimer's disease, premature aging, etc.

A large number of heterocyclic Schiff bases are known to exhibit analgesic, antibacterial antifungal, antiviral antipyretic, cytotoxic, anticancer and, antitumor activities. Some of the Schiff bases are also used as chelating agents.

However despite major efforts, no effective antiglycation agent has been introduced in clinical practices. Therefore, there is an urgent need of the systematic research for the discovery of new effective and safe antiglycation agents as potential drugs for the treatment of late diabetic complications.

### BRIEF SUMMARY OF THE INVENTION

In continuation of our efforts to discover new anti-glycation agents, we synthesized a series of heterocyclic Schiff bases with a variety of heterocyclic aromatic substituents, some of which were earlier reported to have antitumor, bactericidal and pesticidal activities.

In the present investigation, we report the discovery of promising anti-glycation activity of some heterocyclic Schiff bases.

Heterocyclic Schiff bases containing un-substituted and substituted pyridine, benzyl, thiofuran, furan, etc, synthesized through an efficient and high yielding route, exhibited a good antiglycation activity in vitro (FIG. 2). To study the structure-activity relationship, several derivatives of above cited compounds were evaluated for their protein glycation inhibitory activity in vitro. The structure-activity relationship (SAR) studies showed that pyridine containing Schiff base (compound 5) (IC₅₀=397.21±2.2 μM) possess a promising antiglycation activity, when compared with the standard rutin (IC₅₀=294.21±1.5 μM). Compounds 3, 4, and 8 exhibited a moderate inhibition against the protein glycation.

### BRIEF DESCRIPTION OF THE DRAWING

FIG. 1 depicts the structures of heterocyclic Schiff's bases, evaluated for their antiglycation activities.

FIG. 2 depicts the antiglycation activity of heterocyclic Schiff's bases (1-6).

### DETAILED DESCRIPTION OF THE INVENTION

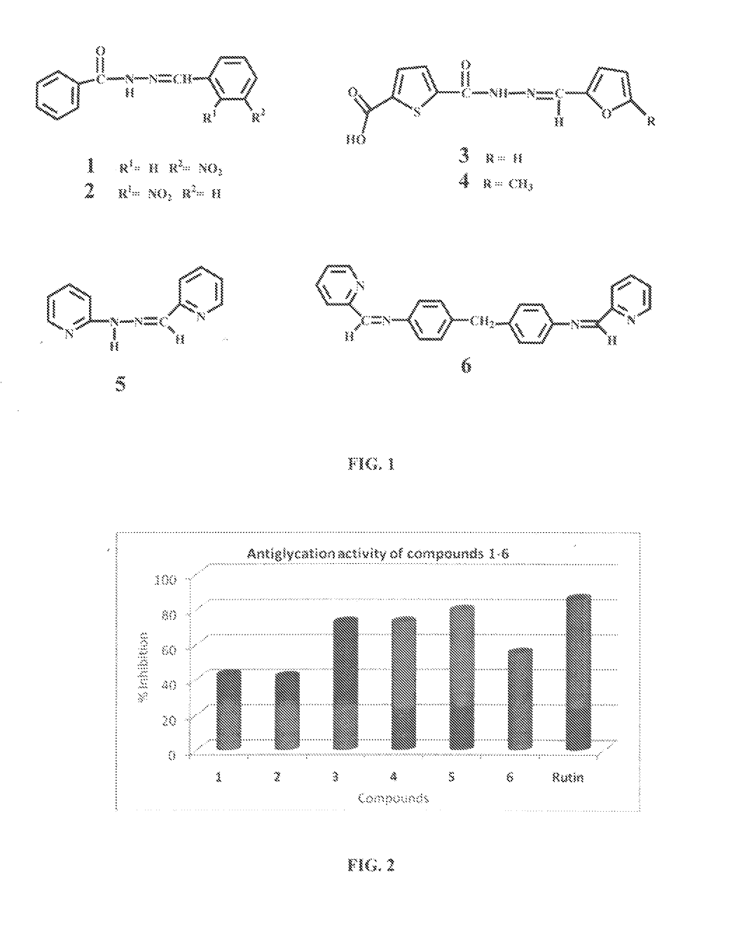

Equimolar amounts of the corresponding aldehydes and hydrazides were mixed together in ethanol. Two drops of concentrated HCl were added to acidify the medium, then the mixture was refluxed for 8 hours, the reaction was monitored by TLC. After cooling, distilled water was added up to 1:3 volume ratio (V_{org}:V_{aq}), followed by addition of several drops of sodium hydroxide solution in order to neutralize the mixture medium. To ensure the purity, the product obtained was re-crystallized. In general, for a typical Schiff base, 80-95% yield was recorded (FIG. 1).

All the chemicals used were of analytical grade and served without further purification as purchased from Sigma-Aldrich (Japan). IR Spectra were recorded by using CHCl₃ solvent. ¹H and ¹³C NMR spectra were recorded at 300 MHz and 75 MHz in CDCl₃, respectively. Mass spectra were obtained at low resolution. All reactions were monitored by thin layer chromatography (TLC) with Merck 60 F₂₅₄ silica gel coated plates.

Bovine Serum Albumin (BSA) was purchased from Merck Marker Pvt. Ltd. (Germany), rutin and methylglyoxal (MG) (40% aqueous solution) were from Sigma Aldrich (Japan), sodium dihydrogen phosphate (NaH₂PO₄), disodium hydrogen phosphate (Na₂HPO₄) and sodium azide (NaN₃) were purchased from Scharlau Chemie, S. A. (Spain), while dimethyl sulphoxide (DMSO) was purchased from Fischer Scientific (UK).

The total reaction volume of the assay was 200 μL, having final concentrations of 10 mg/mL BSA, 14 mM methylglyoxal, and 1 mM test compounds. 10 mg/mL solution of BSA and 14 mM methylglyoxal was prepared in 0.1 M phosphate buffer (pH 7.4), containing sodium azide (NaN₃) (30 mM) as antimicrobial agent, while 1 mM solutions of test compounds were prepared in the DMSO. Assay was performed in triplicate. Each reaction mixtures were comprised of 50 μL BSA, 50 μL methylglyoxal, 20 μL test compound and 80 μL phosphate buffer (pH 7.4). The reaction mixture was incubated under aseptic conditions at 37° C. for 9 days.

After completion of nine days of incubation, each sample was examined for the development of specific fluorescence (excitation 330 nm; emission 420 nm) against blank on a microtitre plate reader (SpectraMax M2, Molecular Devices, CA, USA) [14, 15].

The percent inhibition of AGE formation by the test sample versus control was calculated by using the following formula:

% Inhibition=(2−Fluorescence of test sample/Fluorescence of the control)×100

The IC₅₀ (i.e. the concentration of test samples that inhibit the process of glycation to 50%) was determined by monitoring the effect of various concentrations (ranges from 1000-50 μM) of test compounds. The IC₅₀ values were calculated by using EZ-FIT Enzyme Kinetics Program (Perrella Scientific Inc., Amherst, USA). The antiglycation potential of test compounds was compared with rutin, which was used as standard inhibitor.

Schiff bases 1-6 were evaluated for their antiglycation activity in in vitro BSA-MG glycation model system. These compounds showed IC₅₀ values between 397 and 862 μM (Table-1). Compounds 5 (IC₅₀=397.21±2.2 μM) and 4 (IC₅₀=442.79±0.68 μ) were found to exhibit good antiglycation activity in BSA-MG model assay when compared with standard (rutin, IC₅₀=294.46±1.50 μM). Compounds 3 (IC₅₀=520.83±1.8 μM) and 6 (IC₅₀=862.84±3.3 μM) showed a weak antiglycation potential. While compounds 1 and 2 were found to be inactive, as they showed less than 50% inhibition of protein glycation.

Table 1 shows the results of in vitro antiglycation assay (against bovine serum albumin) on heterocyclic Schiff's bases 1-6.

TABLE 1

| | | Antiglycation |
|---|---|---|
| | | Studies |
| | | IC₅₀ ± SEM |
| Compounds | IUPAC Names | [μM] |
| 1 | N′-(3-Nitrobenzylidene)benzohydrazide | Inactive |
| 2 | N′-(2-Nitrobenzylidene)benzohydrazide | Inactive |
| 3 | 2-Thiophenecarboxylic acid, (2- | 520.83 ± 1.8 |
| | furanylmethylene)hydrazide (9CI) | |
| 4 | 2-Thiophencarboxylic acid, [(5-methyl-2- | 442.79 ± 0.68 |
| | furanyl)methylene] hydrazide (9CI) | |
| 5 | 1,3-Bis(2′-pyridyl)-1,2-diazapropen-2-ene | 397.21 ± 2.2 |
| 6 | Bis[4-(2- | 862.84 ± 3.3 |
| | pyridylmethyleneamino)phenyl]methane | |
| Standard | Rutin | 294.50 ± 1.5 |
| antiglycation | | |

Nitro group of aminoguanidine and other nitrogen containing compounds are well known to form Schiff base adduct with carbonyl compounds or sugars, which is mainly responsible for inhibiting the formation of advanced glycation end product (AGEs). Additionally, it has been found that compounds with different substituents have varying degrees of activity against the protein glycation. This opens a new horizon to understand the mechanism of inhibition. Based on this, we have screened Schiff bases for their antiglycation potential and established a structure-activity relationship. The limited structure-activity relationship suggest that the presence of nitro group apparently has no contribution to inhibit the glycation process, as seen in compounds 1 and 2 possess nitro groups at meta and ortho positions, respectively, and both were found to be inactive.

Compound 3 has a thiophenecarboxylic acid group at one side of the molecule and a furanyl moiety on the other side of the molecule. This compound showed a weak anti-glycation activity with IC₅₀ value of 520.83±1.8 μM, while compound 4 possess thiophenecarboxylic acid group at one side of the molecule and 5-methyl-furanyl moiety on the other side of the molecule, and showed an increased antiglycation activity (IC₅₀=442.79±0.68 μM).

These results suggest that the presence of electron donating group, e.g. methyl group was found to be beneficial for the activity. Compound 5 was found to be the most active analog of the series with IC₅₀ value of 397.21±2.2 μM. This compound has unsubstituted pyridine moiety on both sides of the molecule. It may be assumed that nitrogen atom of pyridine moiety may react with carbonyl moiety of MG, therefore inhibiting its reaction with the amino terminus of the protein. Compound 6 was found to be the weakest analog with IC₅₀ value of 862.84±3.3 μM. This compound, although possess pyridine groups on both side of the derivative, but they are separated by “diphynylmethyl moiety”, hence we can suggest that here electronic factor tend to decrease the antiglycation potential.

Most of the Schiff bases, which inhibited the antiglycation activity, were not found to be toxic in nature 3T3 cell lines. Therefore, they can be used as potential antiglycation agents to prevent late diabetic complications. They have the ability to interact with the post Amadori (Amatory) step to inhibit the formation of advance glycation end products (AGEs).

1. A method of treating advanced glycation endproduct (“AGE”)-related disorders linked to body protein glycation reaction, by administering a suitable amount of 1,3-bis(2′-pyridyl)-1,2-diazapropen-2-ene to animals and humans in need of this treatment.

2. A method of treating AGE related disorders linked to protein glycation reaction by administering a suitable amount of bis[4-(2-pyridylmethyleneamino)phenyl]methane to animals and humans in need of this treatment.
